(19) United States
(12) Patent Application Publication
(10) Pub. No.: US 2009/0048642 A1
(43) Pub. Date: Feb. 19, 2009

(54) NEUROSTIMULATION

(76) Inventor: Teodor Goroszeniuk, London (GB)

Correspondence Address: DENNISON, SCHULTZ & MACDONALD, 1727 KING STREET, SUITE 105, ALEXANDRIA, VA, 22314, US

(21) Appl. No.: 12/280,042

(22) PCT Filed: Feb. 19, 2007
(86) PCT No.: PCT/GB07/00585

§ 371 Date: Aug. 20, 2008

(30) Foreign Application Priority Data
Feb. 21, 2006 (GB) 0603464.9
Nov. 10, 2006 (GB) 0622457.0

(51) Int. Cl. A61N 1/34

(52) U.S. Cl. 607/46

(57) ABSTRACT

The invention relates to a device for treating a patient for chronic pain and for cosmetic treatment by neurostimulation comprising a plurality of stimulating electrodes and one or more reference electrodes to be applied externally, ie to the surface of the skin, in the region of the pain and to apply a current of between 0.2 and 12 mA at a frequency of between 1 and 50 Hz, and preferably between 2 and 10 Hz. Optimal results appear to be achieved when the applied current is between 0.2 and 6 OmA depending on the depth of the pain and the tenderness of the skin of the area to be treated. In use the area to be treated is located either by a stimulating device, or by using at least one of the stimulating electrodes on the device itself to locate the pain prior to treatment. The electrodes are applied to the patient's skin as located using firm pressure whilst a stimulating pulse is applied as treatment. This action produces a remarkable and unexpected level of pain relief over a wide area. The invention extends to a method for the treatment of chronic pain using the above-mentioned parameters and procedures.

This invention relates to improvements in neurostimulation for the treatment of chronic pain and for cosmetic treatment.

Neurostimulation is gaining in popularity as a treatment of chronic pain. Traditionally such neurostimulation has relied on the implantation of a device in a position adjacent to an affected area. An electrical stimulation is applied to implanted electrodes to achieve a level of relief in the patient. However, such devices need to be accurately located and generally require a small surgical procedure in order to implant the device. The procedure is carried out by a trained medical practitioner or specialist.

In my earlier UK patent application published under no. GB2417688 I describe a device for the treatment of chronic pain by electrical neurostimulation comprising a pair of electrodes and an electronic power supply arranged to supply a pulsed signal between the electrodes which are arranged, in use, to be pressed firmly onto a patient's skin both accurately to locate the source of pain in response to a locating pulse and then to remain in place as located to treat the pain by applying an appropriate stimulating pulsed signal transcutaneously to treat the pain.

In accordance with the invention, a stimulating device for the treatment of chronic pain, especially neuropathic and myofacial pain, and for cosmetic purposes by means of external or transcutaneous electrical neurostimulation comprises an electronic power supply and a plurality of electrodes arranged to be applied externally to a patient's skin so that the power supply can apply a pulsed stimulating signal between the electrodes of which at least one electrode is a reference electrode, and the other electrodes are stimulating electrodes which are arranged to apply an appropriate pulsed low frequency stimulating signal transcutaneously to treat the pain.

The fact that the electrodes are arranged to be applied externally for non-invasive use permits the device to be used and, to a certain extent, controlled by a patient himself, although the initial set-up of the device will be performed by a medical practitioner or specialist. The very low current which has been found to be effective allows the device to be manipulated safely by older people—the most frequent sufferers from chronic pain—or even by children or people who are handicapped without endangering them.

The device is most effective when the stimulating electrodes comprise substantially hemispherical contacts which project in such a way that they can be applied to an affected area with sufficient pressure that they produce small indentations in the skin. The electrodes are preferably mounted on a non-conducting, semi-rigid support or supports. The firm pressure, sufficient to produce small indentations in the skin, together with the stimulating signal contributes significantly to the effective treatment of the pain.

In a preferred form of the invention a number of stimulating electrodes is used to treat an extended area. There may be between 2 and 24, though generally not more than 16, stimulating electrodes whose centres are between 50 and 150 mm apart. In practical terms there are ideally 4 to 6 stimulating electrodes with an enclosed area of 150 to 200 cm². For treatment of the back or spine, however, typically 16 electrodes may be required, arranged in 8 pairs and covering up to 1000 cm².

More generally, the electrodes will be mounted on a semi-rigid plastic backing, which may be curved to suit the body part to which it is intended to be applied. Alternatively, the backing containing the cluster of electrodes may be provided with a strap which allows it to be attached and held in place on a patient. This is particularly useful where the arm, wrist or chest is to be treated.

Various other forms may be designed to suit the various sites to be treated. A hand-held device may be provided with a handle having the electrodes at its outer end; the handle may even be in the form of a pistol-like grip. A board populated with electrodes may be used for treating back or spinal pain. Collars or belts are particularly useful in that they can be held firmly in place around a limb, neck or torso. Or the electrodes may be incorporated into a cap, glove or sock to treat a specific location. A larger number of electrodes may be incorporated into such items permitting them to be standardised, albeit in practice only the required number of specific electrodes may be activated in use on a particular patient.

This combination of firm pressure together with the electrical stimulation provides a surprising level of relief comparable to that achieved by a percutaneous implant. Furthermore the multiple electrodes extend the relief over an area which is simply not feasible using any other known method. In a recent trial, in more than half the patients on which the device was used the VAS pain score was reduced to 0 over a period of five minutes.

The present invention must be distinguished from the known transcutaneous electrical nerve stimulation (TENS) in which silicon-rubber/carbon electrodes are stuck onto a patient's skin in the region of an affected or painful area. In the present invention, the stimulating electrodes are fairly accurately located with respect to an affected nerve using a locating stimulator or the stimulating device itself in its locating mode. In addition each stimulating electrode has a hard, rounded end, generally at the end of a rigid stem, which can be 1) applied firmly to the skin in the affected area as located either by the device in a location mode or by a separate nerve locator, and 2) moved in response to the patient's reaction to the stimulating signal applied to the stimulating electrodes further to fine-tune the treatment.

The stimulating signal is advantageously applied to different electrodes or pairs or selected groups of electrodes alternately, sequentially or in rotation to improve the level of relief to the patient, and the power supply is conveniently arranged to offer this feature. This also has the advantage of reducing the current density at the reference electrode(s) and the overall required power output of the power supply.

The stimulating signal applied to each electrode may be applied sequentially with such a short delay between each electrode that it is indiscernible by the patient that not all the electrodes are activated simultaneously.

In general the stimulating electrodes will be anodes (+) with the reference electrode being the cathode (−). However, some patients find that inverting the polarity produces greater relief and thus the polarity can be selected by the specialist accordingly.

The energy required for the treatment may depend on the size of the nerves (large, heavily myelinated A a motor fibres at one extreme versus smaller uimiyelinated C fibres at the other). Thus the energy delivered E (energy in nC)=I (current in mA)×t (duration in μs). In a typical example, a pulse may last from 0.05 ms to 1 ms and the applied current may be from 0.5 mA to 60 mA. In practice the highest currents would not be used with the longest pulse widths, so that the power per pulse would not be likely to exceed 50 mC and would be more likely to be in the order of 15 to 25 mC or less for a tender part of the body.

In order to obtain the necessary transcutaneous current a relatively high voltage—up to 85V, though more traditionally 65V—may be required. The pressure that can be applied by the stimulating electrodes (and by the other, reference, electrode) to the skin allows the voltage to be reduced because of the lower resistance, making burning less likely. The provision of rounded ends of the electrodes also acts to distribute the current more accurately and evenly than would be achieved by a traditional silicon carbon electrode. A gel may be used to enhance the conductivity between the reference electrode and the skin.

In other embodiments one of the electrodes, the reference electrode, may comprise a silicon-carbon patch connected to the power supply and is arranged to be stuck to the skin close to the affected area. In order to avoid an excessive current density at the point of contact with a patient's skin, the area of the reference electrode may be greater than that of a single stimulating electrode if more than a single stimulating electrode is likely to be operated simultaneously.

Additionally, the power supply is arranged so that if one or more of the electrodes or pairs of electrodes does not make proper contact with the patient's skin, the current is accordingly reduced to prevent discomfort or even burning which might otherwise occur.

This external approach whereby the electrical impulse is applied externally over the nerves, plexuses and at non-specific areas in a non-segmental distribution produces results which are overwhelming. Pain relief can be compared to the percutaneous direct approach, with results of the same magnitude of 70-100% pain relief achieved as measured on a VAS score.

The duration of the pain relief following a typical 5 min session where the electrodes have been placed correctly can vary between minutes or hours and days or even weeks.

One of the most important components of this novel approach to peripheral neuromodulation in treatment of neuropathic pain is the frequency of the stimulation, which is contrary to established recommendations. The frequency may be effective in the range of stimulation 1-50 Hz, and preferably between 2 and 10 Hz or up to 50 Hz, but not usually in the 50-150 Hz range.

The device may thus be set to provide a specific current at each stimulating electrode of between 0.2 mA and 60 mA. Optimal results appear to be achieved when the applied current to each electrode is between 2 and 10 mA. The current combined with the pulse width determines the level of energy applied. The pulse width may be from 0.05 to 1 ms. The penetration of the signal increases with the pulse width while the applied current may be increased at shorter pulse widths.

The current is set by the practitioner according to the pain, the patient's skin and the part of the body to be treated with an objective to use the lowest current consistent with achieving the desired relief and without causing discomfort or burning. Clearly more delicate parts of the body, for example around the eyes, or more fragile skin will require the current and power to be limited accordingly.

Once the stimulating electrodes have been accurately located the current can be reduced in most cases and the frequency can be adjusted to achieve the optimum level of relief. Accurate location of the electrodes thus reduces the discomfort to the patient and the risk of burning. It also prolongs battery life for a portable device.

For maximum effectiveness the electrodes are accurately positioned using the device in its stimulator-and-location mode prior to treatment. Thus, the device is first used to position the treatment electrodes prior to fine-tuning the treatment current and frequency. The power supply may then be adjusted and used to provide the desired electrical output to the electrodes for the treatment.

As it may not be necessary to carry out the location procedure on each occasion, either a separate nerve stimulation function on the device may be used by the specialist practitioner who prescribes the treatment, or a separate locating stimulator may be used. In normal use the location function is switched off so that the patient is not confused. The treatment parameters equally may be pre set or pre-limited to prevent the patient from harming himself by mistake.

The diameter of the contacts of the electrodes will depend on the muscle or nerve type to be treated. They may be between 4 and 12 mm long or in some cases up to 20 mm, but generally 4 to 8 mm. They may be mounted on the inner side (in use) of a semi-rigid strap which may be attached to a patient by means of a sticky plaster or a belt or strap secured by a of Velcro (® 3M Corp. Inc.) clasp.

The invention extends to a method of treating a patient for chronic pain or for cosmetic treatment by neurostimulation comprising applying a plurality of electrodes externally, ie. to the surface of the skin, in the region of the pain, applying firm pressure to the electrodes and passing a low frequency current between one or more reference electrodes and the stimulating electrodes.

External multiple neurostimulation in accordance with the invention results in improved relief of chronic neuropathic pain, an improvement in peripheral circulation, improved mobility and improved sensory perception. It has the great advantage that it can be applied by the patient him/herself when required and as often as they wish with little or no adverse effects. As no surgical procedure is required, treatment can be easily be modified or stopped if it fails to produce the desired relief or if it causes an unwelcome response.

Additionally, it can be used in conjunction with traditional analgesics or implants either to complement the relief or if the site of the pain moves. This is particularly useful in the treatment of non-specific or non-segmental pain.

The invention will now be further described by way of example with reference to the accompanying drawing in which:

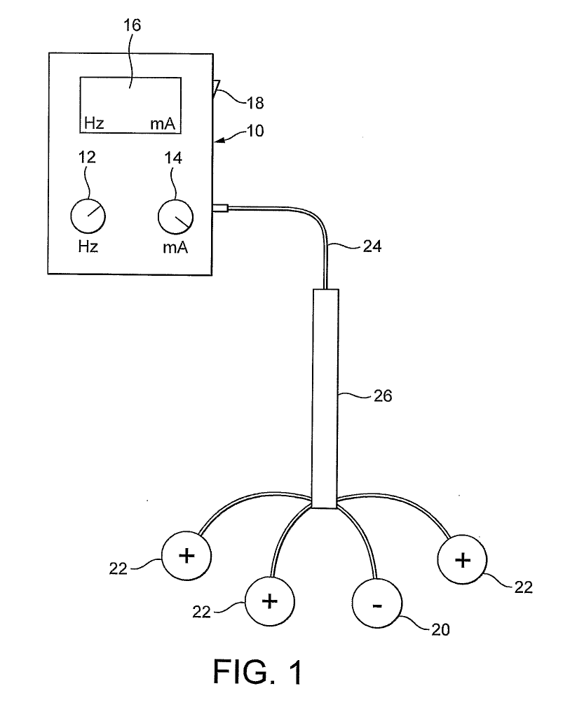

FIG. 1 is a diagrammatic view of a hand-held stimulating device in accordance with the invention;

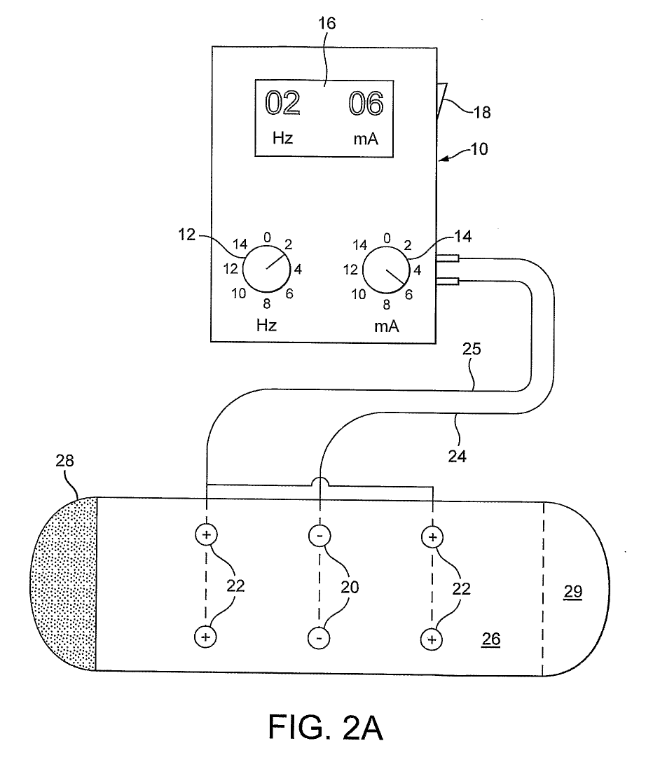

FIG. 2A is a diagrammatic view of a stimulating device where the electrodes are mounted on a flexible, semi-rigid band;

FIG. 2B is a diagrammatic view of a device similar to that in FIG. 2A, but where the reference electrode is in the form of a patch;

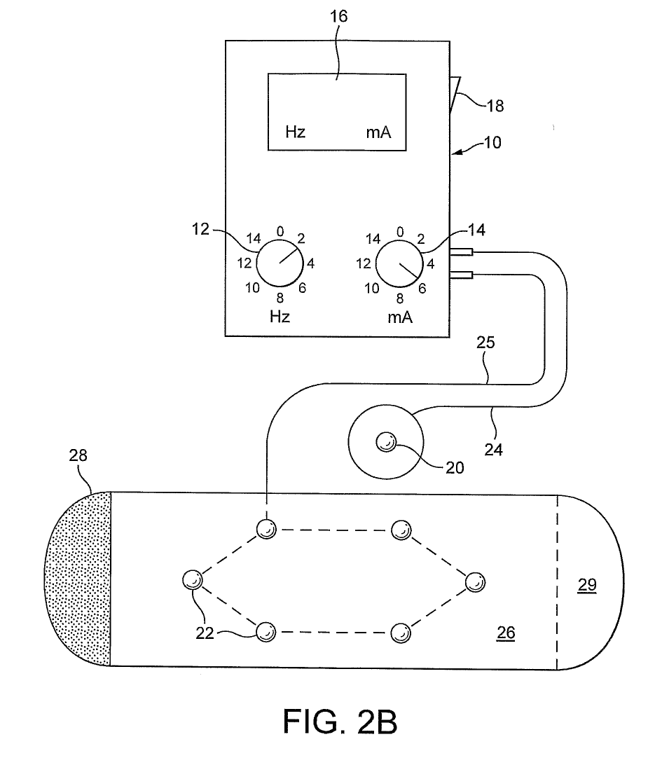

FIG. 3 (A) to (C) are diagrammatic side elevations illustrating possible arrangements of the stimulating electrodes; FIG. 3A is a slightly rounded, hand-held frame which can be moved and held by the patient to treat more generalised pain; FIG. 3B illustrates a back treatment strip, and FIG. 3C shows a curved chest belt;

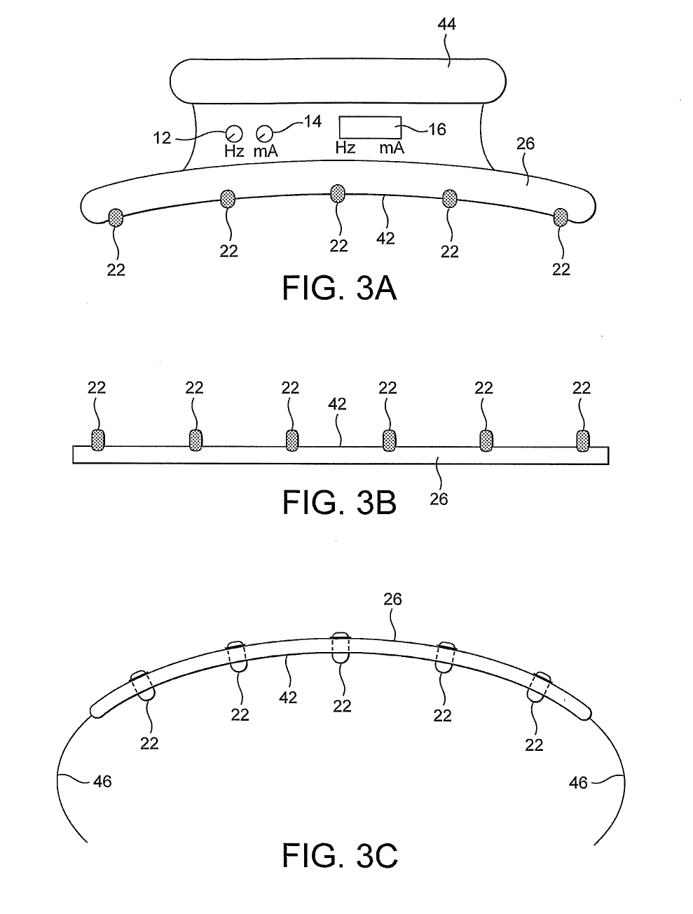

FIG. 4 (A) to (C) illustrate three different geometric arrangements of clusters for stimulating electrodes for use with the device;

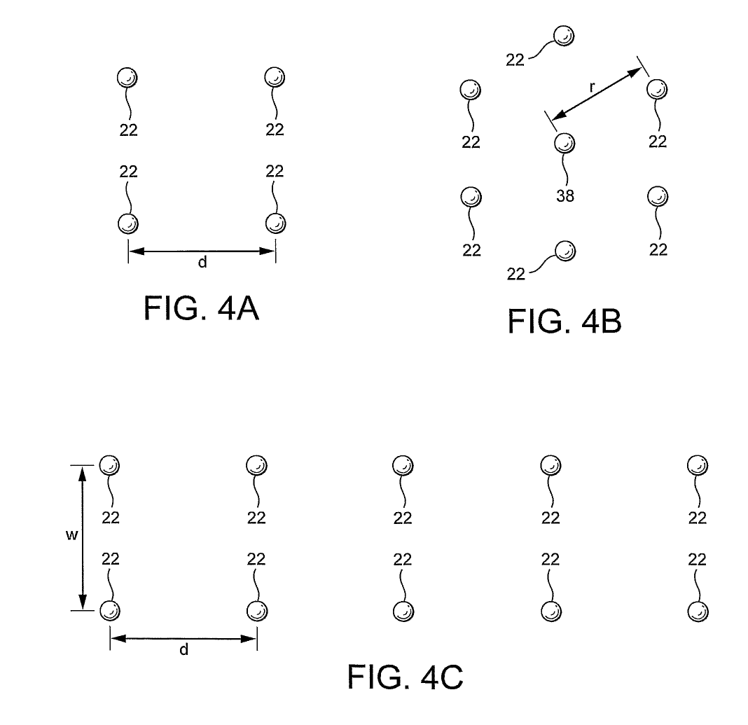

FIG. 5 shows diagrammatically a multi-electrode hand-held probe similar to that shown in FIG. 1;

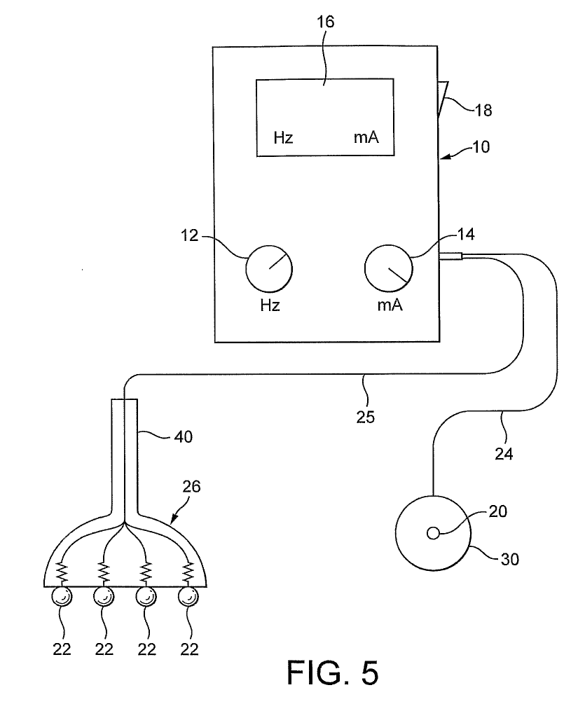

FIGS. 6A and 6B illustrate diagrammatically a cap or headpiece fitted with electrodes;

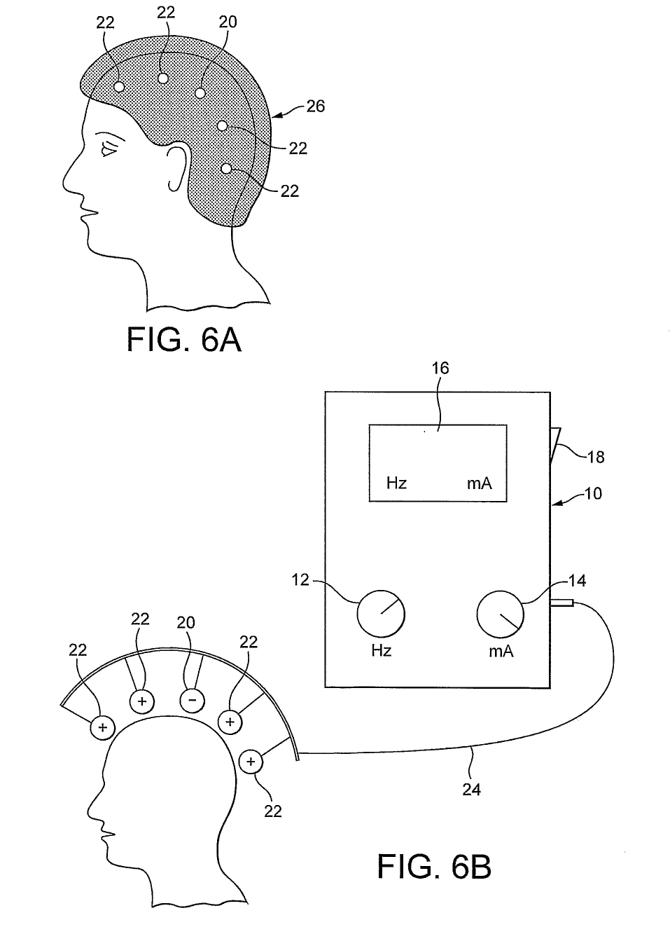

FIG. 7 shows diagrammatically a glove fitted with multiple electrodes; and

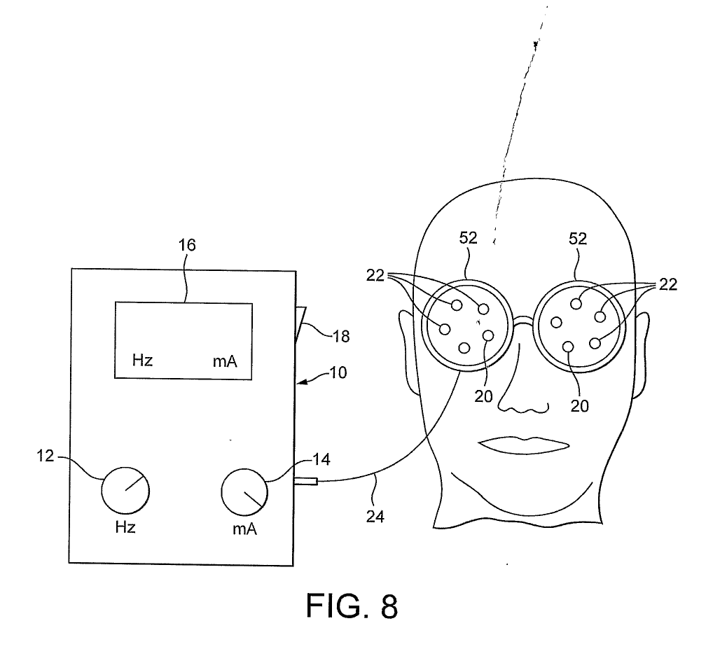

FIG. 9 shows diagrammatically a pair of eye patches fitted with electrodes.

In FIG. 1 the stimulating device comprises a control unit 10 incorporating a power supply which is controlled by a frequency button 12 and a power button 14. The output parameters are displayed on an LCD screen 16. An on/off switch is provided at 18.

The output from the device is supplied by leads 24 to a reference electrode 20 and three stimulating electrodes 22. Each of the electrodes 20, 22 comprises a metal stud with a substantially hemispherical tip projecting from a sturdy support or handle 26 by which a patient can press the electrodes against the skin.

In FIGS. 2A and 2B the electrodes 20, 22 are mounted on a semi-rigid but flexible support 26 that can be attached firmly for example around a patient's wrist or chest by means of a Velcro strip at either end. In FIG. 2A pair of reference electrodes 20 (cathodes) are connected to the stimulating device by a lead 24, and the stimulating electrodes 22 (anodes) are connected by a lead 25. In FIG. 2B the reference electrode 20 is mounted on a separate silicone patch. The number of stimulating electrodes 22 and their spacing will be chosen by the specialist to suit the symptoms of the patient, generally by trial and error.

Each of the electrodes 20, 22 has a diameter of 4 to 8 mm and projects about 5 to 10 mm from a support 26 formed of semi-rigid or stiff, insulating plastics material. They are fixed firmly to the support so that when the latter is attached by means of a strap sufficient pressure can be applied to the electrodes. For example, around a patient's chest, the electrodes should cause an indentation in the patient's flesh and are typically spaced apart by about 100 mm, though they may be customised as the exact required spacing will depend upon the diagnosis and the treatment prescribed by a practitioner.

In practice the centre pair of electrodes 20 (FIG. 2A) acts to produce a contact with the patient's skin as reference electrodes 20 whilst the others, or stimulating electrodes 22, are on either side of the reference electrodes 20. The reference electrodes may be located at the perceived centre of the point of pain in the affected area, in order to produce the optimum level of relief. The stimulating signal acts on both pairs of stimulating electrodes substantially simultaneously, or alternately. The effective area of the reference electrodes 20 may have to be enlarged to reduce the current concentration on the skin if it is intended to activate all the stimulating electrodes simultaneously.

In the device 10 in FIG. 2B the reference electrode 20 is held onto the skin by means of a silicon-carbon patch 30 arranged to be attached to the skin of a patient close to the affected area. A gel may be used to enhance the conductivity between the electrodes and the skin. The stimulating electrodes 22, are arranged here in a hexagonal pattern of a short stems set into a semi-rigid support 26. The support has a strap shown is for attaching around a patient's chest by means of a length of Velcro® 28 at one end which attaches to a corresponding patch 29 on the other end of the strap.

The stimulating electrodes 22 may be arranged in any desired pattern, such as one of those shown and described below with reference to FIG. 4. Once the electrodes have been correctly located, the strap 26 is attached firmly to hold the electrodes against the patient's skin whilst the stimulating signal is adjusted to provide the optimum level of relief whilst minimising any discomfort.

FIG. 3 illustrates diagrammatically four arrangements of the stimulating electrodes. FIG. 3A is a diagrammatic section of a rigid hand-held support 26 having five pairs of stimulating electrodes 22 in two parallel rows. The support has a shallow curve to conform to the shape of the chest and has a handle 44 to allow it to be held firmly in position. The electrodes comprise metal conducting studs about 6 mm in diameter having rounded ends 40 projecting about 8 mm beyond the inner surface 42 of the support 26. Each stud shown represents a pair or more studs in line behind it, as required by the application. A reference electrode (not shown) is provided as above.

FIG. 3B shows a typical support 26 for pairs of stimulating electrodes 22 suitable for treatment of spinal pain. In this case the support may be less rigid than in the supports shown above, and it may be laid on a mattress backing. The patient can then lie on the support during treatment. In this case the electrodes may be slightly shorter, say 5 to 6 mm from the inner surface 42 to prevent undue damage to the skin.

FIG. 3C is a diagrammatic section of a support 26 in the form of a belt or collar which is made of a semi-flexible plastics material which is arranged to conform to a patient's body, chest, limb or neck. At either end of the support 26 there is a strap 46 whose ends can be attached to hold it in place during treatment and to provide the desired pressure between the electrodes and the patient's skin.

The stimulating electrodes 22 may be arranged in any desired pattern, such as any of those shown and described below with reference to FIG. 4.

FIG. 4 shows in plan various patterns that may be used for the electrodes 22. FIG. 4A shows stimulating electrodes 22 in the shape of a simple square for use with a separate reference electrode (not shown). The dimensions are selected to fit the pain pattern of a patient; the distance d between adjacent electrodes may be from about 20 to 150 mm; 100 mm has been found to be very effective for the back or spine whilst the electrodes will be more closely spaced for smaller members, such as the hand, neck or eyes. Each electrode has a rounded contact end substantially in the form of a hemisphere.

FIG. 4B shows a circular or hexagonal array of stimulating electrodes 22 surrounding a central electrode 38 which may be used as the reference electrode. If a patch 30 as shown in FIG. 2 is used, the central electrode 38 will be another stimulating electrode activated with the other electrodes or in sequence. The central electrode 38 may also be used as a locating electrode in order to assist in locating the device. The radius r of the array or cluster will typically be about 100 mm.

FIG. 4C shows an elongated array of electrodes 22 for treating extended spinal pain as described with reference to FIG. 3B. Generally up to 9 pairs of electrodes are sufficient to treat back pain, though more may be used if required.

This electrode array appears to produce good results for such larger sites and may be to about 100 mm in width and spaced apart by distance d which is typically 100 mm. Such larger electrode arrays may comprise up to 24 electrodes, but this may also call for multiple reference electrodes or one having a greater surface area if multiple stimulating electrodes are to be activated simultaneously.

FIG. 5 shows diagrammatically a multi-electrode hand-held support 26 similar to that shown in FIG. 1, and attached to a power supply 10. The support has a number of stimulating electrodes 22 and has a handle 40 to permit it to be held firmly in place against the skin. A separate reference electrode 20 is provided on a silicone patch 30.

FIGS. 6A and 6B show diagrammatically a cap or headpiece 26 fitted with five pairs of stimulating electrodes 22 connected to the power supply 10. One or more reference electrodes 20 is also provided in the headpiece as required.

FIG. 7 shows diagrammatically a glove 50 fitted with multiple electrodes 22 connected to a power source 10. In order to make the glove a standard item that can be adapted to as many patients as possible, a large number of stimulating electrodes 22 are provided which call be activated selectively as required to treat the patient's pain.

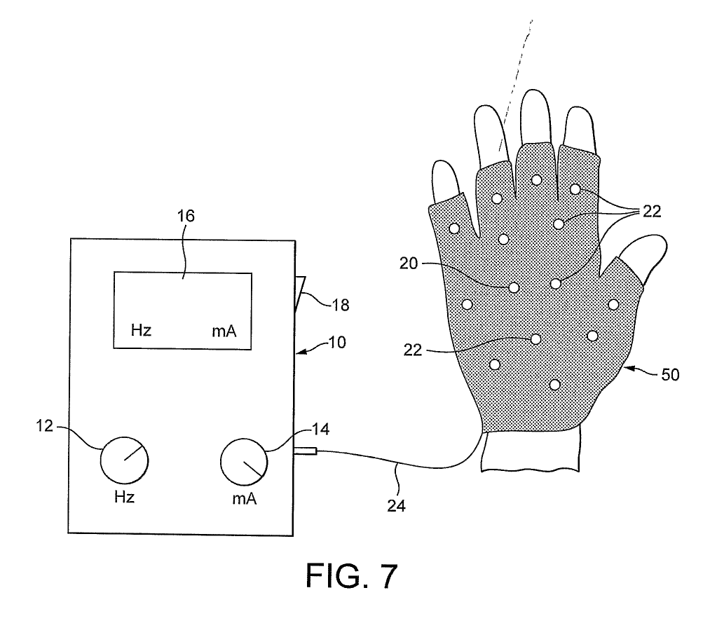

Similarly, FIG. 8 shows diagrammatically a pair of eye shields 52 each fitted with 5 stimulating electrodes 22. The stimulating electrodes 22 can be activated as required from the power supply 10. The reference electrode may be attached separately to the patient, or make use of one of the electrodes on one or both of the eye shields.

Other forms of the device are possible. For example, many sufferers of acute pain have muscular or other disabilities which make it difficult for them to hold the device in a way that applies the appropriate pressure in the desired location. Another form for hand pain, not described here in detail is a pebble or egg-shaped device having an array of electrodes projecting over a portion of its surface. The controls can be protected under a hinged or removable cover.

In use, the combination of the pressure and the, albeit small, low frequency stimulating current appears to have a remarkably beneficial effect in terms of the pain relief achieved.

The power button 14 of the power supply 10 is adjustable typically to provide a current up to the maximum of 60 mA. As described above, the frequency is varied by the frequency button 12. The pulse width may either be varied by means of another control (not shown) or varied automatically as a function of the applied current in order to optimise the applied power. For example, a stimulating pulse in the shape of a square-wave with a frequency of 15 Hz has a pulse with a duration of 0.7 milliseconds at a current of 12 mA may be suitable for treating pain in a wrist or hand. A higher power level (in Coulombs) may be required for back or spinal pain, and rather less for facial or eye pain. This appears to produce an effective treatment whilst enhancing battery life in the case of a portable device. Thus the shorter the pulse and the lower the current the longer the battery life.

As mentioned above in the introduction, the stimulating electrodes are located fairly accurately by means of a locating stimulator, or by using the stimulating device in its locating mode where a signal is sent to one or more of the stimulating electrodes in order to locate the pain to be treated. ‘Fairly accurately’ is a good enough indication of the region as often the patient may not be able to be very precise or specific, but because of the multiple stimulating electrodes incorporated in the stimulating device in accordance with the invention, relief is provided over a larger area than with any other known device.

Once the pain has been located, the locating stimulator is removed, or the locating mode on the stimulating device is disabled. The chosen electrode array is then placed on the site of the pain and the various parameters of the stimulating signal are adjusted by a specialist while the patient indicates the optimum level of relief obtained.

Once the specialist is satisfied, he adjusts the settings of the power supply allowing for some margin and autonomy for the patient to make some minor adjustments to the settings in the light of his experience without further supervision. The patient is then free to use the device as often as he wishes in the knowledge that the device is safe. It is also small enough to be taken with him as required; the low power level used provides a high level of autonomy and independence in case the pain flares up unexpectedly.

Whilst treatment of chronic pain and for cosmetic purposes using a device in accordance with the invention would in all probability have been achieved using an implanted device, the success of this non-invasive, multiple external treatment is astonishing. The flexibility and convenience of this multiple external stimulation device and method is thus greatly appreciated by patients and specialists alike.

1. A stimulating device for the treatment of chronic pain by means of external or transcutaneous electrical neuro-stimulation comprising an electronic power supply which in use is connected to a plurality of electrodes arranged to be applied externally to a patient's skin so that the power supply can apply a pulsed signal between the electrodes of which at least one electrode is a reference electrode, and the other stimulating electrodes at a low frequency, pulsed stimulating signal transcutaneously to treat the pain.

2. A device as claimed in claim 1 in which the power supply is adapted to be able to apply a locating pulse to one or more of the stimulating electrodes in order to locate the source of pain in a patient.

3. A device as claimed in claim 1 in which there are between 2 and 24, and preferably between 4 and 6, stimulating electrodes.

4. A device as claimed in claim 1 in which the stimulating electrodes comprise metallic studs having rounded outer ends, mounted on a semi-rigid insulating support.

5. A device as claimed in claim 1 in which the stimulating electrodes comprise spring mounted balls or plungers.

6. A device as claimed in claim 4 in which the studs, balls or plungers project from their support by between 4 and 12 mm, and preferably by 4 to 8 mm.

7. A device as claimed in claim 1 in which each stimulating electrode has a diameter of between 3 and 12 mm, and preferably between 3 and 5 mm.

8. A device as claimed in claim 1 in which the enclosed area of the stimulating electrodes is between 100 and 1000 cm².

9. A device as claimed in claim 1 in which the enclosed area of the stimulating electrodes is between 150 and 300 cm².

10. A device as claimed in claim 1 in which the reference electrode comprises a separate gel-type patch for attaching to the skin of a patient.

11. A device as claimed in claim 1 in which the reference electrode has a greater surface area than any one of the stimulating electrodes.

12. A device as claimed in claim 1 in which the stimulating electrodes surround the reference electrode.

13. A device as claimed in claim 1 in which the stimulating electrodes are mounted on a rigid or semi-rigid block provided with a handle or strap to enable it to be held firmly in position.

14. A device as claimed in claim 12 in which the device is provided with an integral strap so that it can be attached to a patient.

15. A device as claimed in claim 1 in which the power supply is arranged to deliver a pulsed signal at between 1 and 50 Hz, and preferably between 2 and 10 Hz.

16. A device as claimed in claim 1 in which the power supply is arranged to operate at a voltage of 50 to 85 volts, and preferably at approximately 65 volts.

17. A device as claimed in claim 1 in which the power supply can be arranged to supply a current of between 0.2 mA and 60 mA and preferably between 2 and 10 mA per electrode when the electrodes are applied externally to the skin of a patient.

18. A device as claimed in claim 1 in which the power supply is capable of delivering a pulse duration of between 0.05 ms and 1 ms.

19. A device as claimed in claim 1 in which the power supply is capable of delivering between 1 and 30 mC and preferably between 5 and 15 mC per pulse.

20. A device as claimed in claim 1 in which the power supply is arranged to be capable of applying the stimulating signal to different electrodes or pairs or selected groups of electrodes alternately or in rotation to improve the level of relief to the patient.

21. A device as claimed in claim 1 in which the electrodes are placed on one surface of the body housing the power supply, and straps are provided for holding device in place on a patient as required.

22. A device as claimed in claim 1 comprising a pistol grip to allow a person to hold it firmly in order to apply pressure to the stimulating electrodes at its extremity.

23. (canceled)

24. A method for treating chronic or acute pain by neurostimulation comprising locating the affected nerve or muscle by means of electrical neurostimulation and positioning a plurality of stimulating electrodes externally over the said nerve, applying firm pressure to the electrodes and passing a low frequency electrical stimulating pulse between the stimulating electrodes and one or more reference electrodes.

25. A method as claimed in claim 24 in which a current of between 0.2 and 60 mA is applied to each stimulating electrode at a frequency of between 1 and 50 Hz.

26. A method as claimed in claim 24 where the applied frequency is between 5 and 30 Hz.

27. A method as claimed in claim 24 where the applied pulse-width is between 0.05 ms and 1 ms.

28. (canceled)
